(19) United States
(12) Patent Application Publication
(10) Pub. No.: US 2009/0263363 A1
(43) Pub. Date: Oct. 22, 2009

(54) Compositions and Methods For Treating Bone Formation Disorders

(75) Inventor: Ming Zhang, Glencoe (IL)

Correspondence Address: Casimir Jones, S.C., 440 Science Drive, Suite 203, Madison, WI, 53711, US

(73) Assignee: Northwestern University, Evanston, IL (US)

(21) Appl. No.: 12/428,103

(22) Filed: Apr. 22, 2009

Related U.S. Application Data
(60) Provisional application No. 61/047,018, filed on Apr. 22, 2008.

(51) Int. Cl. A61K 35/12; A61K 38/17; C12N 5/06; A61P 19/10

(52) U.S. Cl. 424/93.7; 514/12; 435/375

(57) ABSTRACT

The present invention relates to compositions and methods for the detecting, treating, and empirically investigating disorders associated with bone formation (e.g., osteoporosis). In particular, the present invention provides compositions and methods for using maspin and targets of maspin in the diagnosis, treatment, and empirical investigation of disorders associated with bone formation (e.g., osteoporosis).

### CROSS-REFERENCE TO RELATED APPLICATIONS

The present application claims priority to U.S. Provisional Patent Application Ser. No. 61/047,018, filed Apr. 22, 2008, which is herein incorporated by reference in its entirety.

### STATEMENT REGARDING FEDERALLY SPONSORED RESEARCH OR DEVELOPMENT

This invention was made with government support under R01CA079736 awarded by the National Institutes of Health. The government has certain rights in the invention.

### FIELD OF THE INVENTION

The present invention relates to compositions and methods for the detecting, treating, and empirically investigating disorders associated with bone formation (e.g., osteoporosis). In particular, the present invention provides compositions and methods for using maspin and targets of maspin in the diagnosis, treatment, and empirical investigation of disorders associated with bone formation (e.g., osteoporosis).

### BACKGROUND OF THE INVENTION

Osteoporosis is a disease of bone that leads to an increased risk of fracture. In osteoporosis the bone mineral density (BMD) is reduced, bone microarchitecture is disrupted, and the amount and variety of non-collagenous proteins in bone is altered. Osteoporosis is defined by the World Health Organization (WHO) in women as a bone mineral density 2.5 standard deviations below peak bone mass (20-year-old healthy female average) as measured by dual energy x-ray; the term “established osteoporosis” includes the presence of a fragility fracture (see, e.g., WHO (1994). “Assessment of fracture risk and its application to screening for postmenopausal osteoporosis. Report of a WHO Study Group”. World Health Organization technical report series 843: 1-129; herein incorporated by reference in its entirety). Osteoporosis is most common in women after menopause, when it is called postmenopausal osteoporosis, but may also develop in elderly men, and may occur in anyone in the presence of particular hormonal disorders and other chronic diseases or as a result of medications, specifically glucocorticoids, when the disease is called steroid- or glucocorticoid-induced osteoporosis (SIOP or GIOP). Given its influence on the risk of fragility fracture, osteoporosis may significantly affect life expectancy and quality of life.

Improved methods for treating, detecting, and empirically researching osteoporosis and disorders associated with bone formation are needed.

### SUMMARY OF THE INVENTION

Experiments conducted during the development of embodiments for the present invention demonstrated promotion of osteoblastic cell proliferation following exposure to maspin. In particular, it was shown that the maspin protein acts on osteoblastic cell surfaces to promote cell proliferation. Accordingly, the present invention relates to compositions and methods for the detecting, treating, and empirically investigating disorders associated with bone formation (e.g., osteoporosis). In particular, the present invention provides compositions and methods for using maspin and targets of maspin in the diagnosis, treatment, and empirical investigation of disorders associated with bone formation (e.g., osteoporosis).

In certain embodiments, the present invention provides methods for treating bone formation disorders. The present invention is not limited to particular methods for treating bone formation disorders. In some embodiments, the methods comprise administering to a subject suffering from a bone formation disorder a composition comprising maspin. The methods are not limited to a particular type or severity of a bone formation disorder. Examples of bone formation disorders include, but are not limited to, osteoporosis. The methods are not limited to a particular type of composition comprising maspin. In some embodiments, the composition comprises maspin expressing TM40-Mp cells. In some embodiments, the composition comprises recombinant maspin. The methods are not limited to treating a certain type of subject. In some embodiments, the subject is a rodent (e.g., mouse), while in other embodiments, the subject is a human being. In some embodiments, the composition is coadministered with, for example, an anti-osteoporosis agent (e.g., a hormone replacement therapy agent, a bisphosphonate (e.g., alendronate (e.g., FOSAMAX)), vitamin D, an androgen, a parathyroid hormone, a selective estrogen-receptor modulators, and a calcitonin-salmon).

In certain embodiments, the present invention provides methods for preventing formation of a bone formation disorder, comprising administering to a subject at risk for developing a bone formation disorder (e.g., osteoporosis) a composition comprising maspin. The methods are not limited to a particular type of composition comprising maspin. In some embodiments, the composition comprises maspin expressing TM40-Mp cells. In some embodiments, the composition comprises recombinant maspin. The methods are not limited to treating a certain type of subject. In some embodiments, the subject is a geriatric human being.

In certain embodiments, the present invention provides methods for promoting osteoblast cell proliferation comprising administering to a sample comprising osteoblast cells a composition comprsing maspin.

In certain embodiments, the present invention provides compositions and methods for using maspin and targets of maspin in the diagnosis, treatment, and empirical investigation of osteolytic bone lesions resulting from decreased osteoblast activity (e.g., related to breast cancer and/or other types of cancer). Maspin is a unique serpin with a tumor-suppressing function in breast cancer (see, e.g., Sheng, S., et. al., (1996) PNAS 93, 11669-11674; Zou, Z., et al., (1994) Science 263, 526-529; each herein incorporated by reference in their entireties). Maspin protein is present both in cytoplasm and on cell surface. Intracellular maspin induces tumor cell apoptosis in response to the stress, whereas cell surface maspin inhibits cell migration and invasion. Breast tumor cells exhibit a high frequency of metastasis to bone. Experiments conducted during the course of developing embodiments of the present invention determined that maspin plays an inhibitory role against breast cancer bone metastasis (see, e.g., Examples V through XIII) through, for example, increasing osteoblast cell proliferation, decreasing RANKL expression, and increasing cyclin D1 expression. Accordingly, in some embodiments, the present invention provides compositions and methods for preventing and/or treating bone lesions (e.g., bone lesions resulting from metastasizing breast cancer) in a subject through administration of maspin (e.g., a therapeutically effective amount of maspin). In some embodiments, administration of maspin results in decreased RANKL expression levels. In some embodiments, administration of maspin results in increased cyclin D1 expression levels.

### BRIEF DESCRIPTION OF THE DRAWINGS

FIG. 1 shows that maspin promotes new bone formation in a mouse model. Representative radiographs for bone lesions from mice were inoculated by the intracardiac injection of mouse mammary TM40D and maspin-overexpression TM40D-Mp tumor cells. A, A typical image of the long bone from a normal control mouse. B, A representative image of bone lesions from mice inoculated with TM40D tumor cells, C. Representative image of bone lesions from mice inoculated with TM40D-Mp cells.

FIG. 2 shows histological analysis of bone lesions from mice inoculated with TM40D and TM40D-Mp tumor cells. Panel A-B, Typical H&E sections of the long bone of normal mouse. Panel C-D, Representative section of bone lesions from mice inoculated with TM40D tumor cells. Arrow indicates the area of tumor cells. Panel E-F, Representative section of bone lesions from mice inoculated with TM40D-Mp cells. Significant new bone formation was observed. Arrowheads indicate novel bone matrix; Arrow indicated the tumor cells. Micrographs shown in the top panels (A, C, and E) were taken at a low magnification (X 25); and the rest (panel B, D, and F) were taken at a high magnification (X 200).

FIG. 3 shows effects of maspin on osteoblast proliferation and new bone formation in an organ culture model. A, Calvariae were removed from 4 days-old BABL/c new-born mice. The calvariae were either treated with or without concentrated conditional medium from TM40D and maspin-overexpression TM40D-Mp cells or treated with recombinant GST-Mp or GST-Mp with or without an anti-maspin antibody for 7 days. H&E staining of representative images were shown. Arrows indicate osteoblast cells. Arrowhead indicates new bone area. B, Quantitation of the average number of osteoblasts under different treatments listed in A. Three randomly selected fields at the same pixel size were viewed under the microscope (X200), and the number of defined osteoblasts was counted. The average number of osteoblasts was analyzed using a statistical program. *P=0.03; **P<0.01; ***P=0.011.

FIG. 4 shows effects of maspin on osteoblats proliferation and differentiation. A, Osteoblast cells were co-cultured with TM40D and TM40D-Mp cells for 48 hours. DNA synthesis in osteoblasts was assessed by 3H-Thymidine incorporation. Data were obtained from triplicate experiments. Values are the mean±SD. *P<0.01; **P<0.01; ***P<0.05. B, Effect of maspin on osteoblastic cell differentiation analyzed by alkaline phosphatase activity assay. Osteoblastic cells treated with TM40D-Mp cells had a significant increased alkaline phosphatase activity compared to that were treated with TM40D cells. Data were from triplicate experiments. Values are the mean±SD. *P=0.1; **P=0.03; ***P=0.03.

FIG. 5 shows a phenotypic comparison of mice that were inoculated with TM40D cells and TM40D-Mp cells. A, Changed body weight in mice inoculated with two different tumor cells. At the endpoint of the experiment, most of the TM40D cells inoculated mice lost weight markedly compared to TM40D-Mp cells inoculated mice. Differences in body weight between these two groups of mice were compared using the Student's t-test (two-sided analysis). All data shown represent the mean±SEM (P <0.01). B. Incidence of bone metastasis. Intracardiac injection of TM40D cells resulted in the development of bone lesions in 60% ( 9/15) of mice. However, only 25% ( 4/16) of TM40D-Mp inoculated mice developed bone lesions (Chi square, P<0.05). C. Survival cures of mice inoculated with TM40D and TM40D-Mp cells. The mice with TM40D cells had a decreased rate of survival compared to the mice inoculated with TM40D-Mp cells (P<0.05). D, Transendothelial invasion assay. ³⁵S-labeled TM40D and TM40D-Mp cells (1×10⁴) were seeded on endothelial cell monolayers that were previously plated on a fibronectin-coated polycarbonate membrane insert with 8.0 μm pores in a chemotaxis chamber, and incubated for 20 hr. The cells that migrated to the bottom of lower chamber were harvested and subjected to quantitation using a liquid scintillation counter. The percentage of invasive cells is calculated by dividing the CPM of cells that migrated to the bottom chamber with the CPM of the initially plated cells. Data were analyzed from triplicate samples (p<0.01).

FIG. 6 shows representative radiographs for bone lesions from the intracardiac injection of mouse mammary TM40D and TM40D-Mp tumor cells. A, A typical image of the long bone from a normal control mouse. B, A representative image of bone lesions from mice inoculated with TM40D tumor cells. Arrowhead indicates the presence of osteolytic lesions, resulting from increased bone resorption. C, Representative images of bone lesions from mice inoculated with TM40D-Mp cells. Arrows indicate increased new bone formation, and arrowhead indicates an osteolytic lesion. D, Radiographic analysis of osteolytic bone lesions in TM40D cells and TM40D-Mp cells-inoculated mice. Lesion areas were measured using NIH Image software from long bone of hindlimbs. Values represent the mean±SEM. P=0.02.

FIG. 7 shows histological analysis of bone lesions from mice inoculated with TM40D and TM40D-Mp tumor cells. Panel A-B, Typical H&E sections of the long bone of normal mouse. Panel C-D, Representative section of bone lesions from mice inoculated with TM40D tumor cells. Note the loss of trabecular bone and the occupancy of bone marrow cavity by tumor cells. Arrow indicates the area of tumor cells. Panel E-F, Representative section of bone lesions from mice inoculated with TM40D-Mp cells. Significant new bone formation was observed. Arrowheads indicate novel bone matrix; Arrow indicated the tumor cells. Micrographs shown in the top panels (A, C, and E) were taken at a low magnification (X 25); and the rest (panel B, D, and F) were taken at a high magnification (X 200).

FIG. 8 shows analysis of osteoclastic lesions in mice inoculated with tumor cells. A, Representative H& E image of osteoclastic lesion from mice inoculated with TM40D tumor cells. Arrows indicate osteoclasts (panel a). TRAP (tartrate-resistant acid phosphatase) staining positive cells were present covering the bone surface adjacent to the tumor cells (panel b). B, Comparison of the average number of osteoclasts in mice with bone lesions from the inoculation of TM40D and TM40D-Mp tumor cells. Five randomly selected fields at the same pixel size were viewed for each slide under the microscope (X 200) and the number of defined osteoclasts was manually counted. Data were analyzed by a student t-Test (P=0.027).

FIG. 9 shows osteoclastogenesis analyses of co-cultured bone marrow and breast cancer cells. A, Bone marrow cells only (control). B, Bone marrow cells plus TM40D cells. C, Bone marrow cells plus TM40D-Mp cells. TRAP (+) MNC cells containing 2 or more nuclei were defined as osteoclasts. Arrows indicate typical osteoclast cells. D, Five randomly selected fields at the same pixel size for each well were observed under the microscope (X200) and the number of defined osteoclasts was manually counted. Data were collected from five independent fields. Triplicate experiments were performed and data analyzed using a statistical program (*P<0.01, **P<0.01).

FIG. 10 shows effects of maspin on osteoblats proliferation and differentiation. A, Osteoblast cells were co-cultured with TM40D and TM40D-Mp cells for 48 hours. DNA synthesis in osteoblasts was assessed by ³H-Thymidine incorporation. Data were obtained from triplicate experiments. Values are the mean±SD. *P<0.01; **P<0.01; ***P<0.05. B, Effect of maspin on osteoblastic cell differentiation analyzed by alkaline phosphatase activity assay. Osteoblastic cells treated with TM40D-Mp cells had a significant increased alkaline phosphatase activity compared to that were treated with TM40D cells. Data were from triplicate experiments. Values are the mean±SD. *P=0.1; **P=0.03; ***P=0.03.

FIG. 11 shows effects of maspin on osteoblast proliferation and new bone formation in an organ culture model. A, Calvariae were removed from 4 days-old BABL/c new-born mice. The calvariae were either treated with or without concentrated conditional medium from TM40D and TM40D-Mp cells or treated with recombinant GST-Mp or GST-Mp with or without an anti-maspin antibody for 7 days. H&E staining of representative images were shown. Arrows indicate osteoblast cells. Arrowhead indicates new bone area. B, Quantitation of the average number of osteoclasts under different treatments listed in A. Three randomly selected fields at the same pixel size were viewed under the microscope (X200), and the number of defined osteoclasts was counted. The average number of osteoclasts was analyzed using a statistical program. *P=0.03; **P<0.01; ***P=0.011.

FIG. 12 shows semi-quantitative RT-PCR for the expression of OPN, PTHrP, OPG in TM40D and TM40D-Mp cells. A, Levels of OPN, PTHrP and OPG expression in tumor cells. The PCR products of three different amplification cycle repetition were shown. L19 was used as an internal control. B, Quantitation of the expression level of OPN in TM40D and TM40D-Mp cells. OPN gene expression in TM40D cells was significantly higher than that in TM40D-Mp cells (P=0.015). C, Comparison of PTHrP levels between TM40D-control cells and TM40D-Mp cells. No significance was shown (P=0.07). D, Comparison of the expression level of OPG between TM40D-control cells and TM40D-Mp cells. P=0.07.

FIG. 13 shows RT-PCR analyses of the levels of RANKL and Cyclin D1 expression in MC3T3-E1 cells treated by using conditioned medium from TM40D and TM40D-Mp cells. A, Levels of RANKL and Cyclin D1 expression in MC3T3-E1 cells. The PCR products of three different amplification cycle repetition were shown. L19 was used as an internal control. B, The level of RANKL expression in MC3T3-E1 cells treated by using CM from TM40D-Mp cells was significantly inhibited as compared with that of MC3T3-E1 cells with the treatment of CM from TM40D cells (P=0.026). C, Cyclin D1 gene expression in MC3T3-E1 cells was inhibited by the treatment with CM from TM40D cells (P=0.04), while this inhibitory effect was reversed by the treatment with CM from TM40D-Mp cells (P=0.043).

FIG. 14 shows that maspin controls osteoclast formation through inhibiting RANKL-mediated signal transduction. Monocytes were cultured by themselves or in the presence of RANKL ligand at 50 and 100 ng/ml for 7 days. Note the differentiation of monocytes to osteclast cells. In the presence of maspin at various dosages, this process is inhibited. At 10 μg/ml, maspin completed inhibits the formation of osteoclast cells.

### DETAILED DESCRIPTION OF THE INVENTION

Bone is a metabolically active and highly organized connective tissue. The main functions of the bones are provision of mechanical and structural support, maintaining blood calcium levels, supporting haematopoiesis and housing the important vital organs such as brain, spinal cord and heart. To accomplish these functions bone needs continuous remodeling. Bone contains two distinct cell types, the osteoblasts, essential for bone formation (synthesis); and the osteoclasts, essential for bone resorption (break down). Morphogenesis and remodeling of bone involves the synthesis of bone matrix by osteoblasts and coordinated resorption by osteoclasts. The co-ordination between the osteoblasts and osteoclasts is very crucial in maintaining bone homeostasis and structural integrity of the skeleton. Both these processes are influenced by several hormones and local factors generated within bone and bone marrow, resulting in a complex network of control mechanisms. An imbalance of osteoblast and osteoclast functions can result in skeletal abnormalities characterized by increased or decreased bone mass. This may leads to excessive bone loss that reflects the balance of bone formation and bone resorption. Bone destruction or resorption is carried out by haematopoietically derived osteoclasts. Their number and activity is determined by cell lineage allocation, proliferation and differentiation of osteoclast precursors and the resorptive efficiency of mature osteoclasts. Relevant bone diseases include osteoporosis, rheumatoid arthritis, and Paget's disease of bone. In these disorders bone resorption exceeds bone formation resulting in decreased skeletal mass. This causes bones to become thin, fragile and susceptible to fracture. The consequences of osteoporotic bone fractures include chronic pain in bone, body deformity including height loss and muscle weakness.

Experiments conducted during the development of embodiments for the present invention demonstrated promotion of osteoblastic cell proliferation following exposure to maspin. In particular, it was shown that the maspin protein acts on osteoblastic cell surfaces to promote cell proliferation. In addition, it was shown that maspin inhibits RANKL expression, increases cyclin D1 expression, and increases osteoblast cell proliferation. Accordingly, the present invention relates to compositions and methods for the detecting, treating, and empirically investigating disorders associated with bone formation (e.g., osteoporosis). In particular, the present invention provides compositions and methods for using maspin and targets of maspin in the diagnosis, treatment, and empirical investigation of disorders associated with bone formation (e.g., osteoporosis). Exemplary compositions and methods of the present invention are described in more detail in the following sections: I. Maspin Polypeptides; II. Detection of Disorders Associated with Bone Formation; III. Therapeutics; IV. Pharmaceutical Compositions; V. Drug Screening; and VI. Kits.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is incorporated herein by reference in their entireties.

### I. Maspin Polypeptides

The serine protease inhibitor superfamily (serpins) is categorized to inhibitory and noninhibitory serpins (see, e.g., Gettins P G (2002) Chem Rev 102:4751-804; herein incorporated by reference in its entirety). Inhibitory serpins use their reactive center loop to trap the target proteinase and inhibit its activity (see, e.g., Gettins P G (2002) Chem Rev 102:4751-804; herein incorporated by reference in its entirety). The noninhibitory serpins have shorter NH2 and COOH termini, and they also lack the classic serpin secretory signal peptide (see, e.g., Gettins P G (2002) Chem Rev 102:4751-804; herein incorporated by reference in its entirety). Recent studies indicate serpins function beyond their serpin properties: they are involved in cell adhesion and play a role in extracellular matrix remodeling (see, e.g., Mikus P, et al., (1996) J Biol Chem 271:10048-53; herein incorporated by reference in its entirety).

Maspin (mammary serine protease inhibitor) (see, e.g., Genbank Accession No.: U54705; Genbank Accession No. U04313; NCBI Reference Sequence: NM_002639.4; U.S. Pat. Nos. 6,933,105, 5,905,023; each herein incorporated by reference in their entireties) shares sequence homology with inhibitory serpins, such as plasminogen activator inhibitors 1 and 2, a-1 anti-trypsin, and noninhibitory serpin ovalbumin (see, e.g., Zou Z, et al., (1994) Science 263:526-9; herein incorporated by reference in its entirety), and several sequence comparisons suggest that maspin is more closely related to noninhibitory clade B serpins. The reactive center loop of maspin is significantly shorter than that of most inhibitory serpins, and maspin does not undergo conformational rearrangement required to inactivate target protease(s) (see, e.g., Pemberton P A., et al., (1995) J biol Chem 270:15832-7; herein incorporated by reference in its entirety). However, the reactive center loop of maspin is important for its function; studies using synthetic maspin reactive center loop peptides and maspin/ovalbumin chimeras reveal that this region is important for promoting cell adhesion (see, e.g., Ngamkitidechakul C, et al., (2003) J biol Chem 278:31796-806; herein incorporated by reference in its entirety).

Maspin has been shown to have tumor-suppressing function in breast cancer. Functional studies have demonstrated that maspin inhibits tumor invasion and motility of human mammary tumor cells in cell culture (see, e.g., Sheng, S., et. al., (1996) PNAS 93, 11669-11674; herein incorporated by reference in its entirety), as well as tumor growth and metastasis in the nude mice assay (see, e.g., Zou, Z., et al., (1994) Science 263, 526-529; herein incorporated by reference in its entirety). The specific expression of maspin in normal mammary epithelial cells, but not in mammary carcinoma cell lines, was shown by Northern blot analysis. This regulation is controlled at the transcriptional level by the combination of elements including Ets and Ap1 elements in breast cells (see, e.g., Zhang, M., et al., (1997) Cell Growth Differ. 8, 179-186; herein incorporated by reference in its entirety).

In experiments conducted during the development of embodiments for the present invention, maspin was shown to promote osteoblastic cell proliferation. In particular, it was shown that the maspin protein acts on osteoblastic cell surfaces to promote cell proliferation. Accordingly, in some embodiments, the present invention provides compositions comprising maspin for purposes of treating and empirically investigating disorders associated with bone formation (e.g., osteoporosis). In some embodiments, the present invention provides compositions comprising functional equivalents of maspin and/or variants of maspin (see, e.g., U.S. Pat. Nos. 5,905,023, 5,801,001, 5,470,970; each herein incorporated by reference in their entireties) for purposes of treating and empirically investigating disorders associated with bone formation (e.g., bone lesions (e.g., bone lesions resulting from breast cancer), osteoporosis).

In preferred embodiments, variants of maspin result from polymorphisms or mutations (i.e., a change in the nucleic acid sequence) and generally produce altered mRNAs or polypeptides whose structure or function may or may not be altered. Any given gene may have none, one, or many variant forms. Common mutational changes that give rise to variants are generally ascribed to deletions, additions or substitutions of nucleic acids. Each of these types of changes may occur alone, or in combination with the others, and at the rate of one or more times in a given sequence.

It is contemplated that it is possible to modify the structure of a maspin peptide having a function (e.g., osteoblast cell proliferation function) for such purposes as altering the biological activity (e.g., altered osteoblast cell proliferation function). Such modified peptides are considered functional equivalents of peptides having an activity of a maspin peptide as defined herein. A modified peptide can be produced in which the nucleotide sequence encoding the polypeptide has been altered, such as by substitution, deletion, or addition. In particularly preferred embodiments, these modifications do not significantly reduce the biological activity of the modified maspin peptides. In other words, construct “X” can be evaluated in order to determine whether it is a member of the genus of modified or variant maspin's of the present invention as defined functionally, rather than structurally. In preferred embodiments, the activity of variant maspin polypeptides is evaluated by methods described herein. In some embodiments, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of maspin containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (see, e.g., Stryer ed., Biochemistry, pg. 17-21, 2nd ed, WH Freeman and Co., 1981; herein incorporated by reference in its entirety). Whether a change in the amino acid sequence of a peptide results in a functional polypeptide can be readily determined by assessing the ability of the variant peptide to function in a fashion similar to the wild-type protein. Peptides having more than one replacement can readily be tested in the same manner.

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

In some embodiments, maspin variants are produced by methods such as directed evolution or other techniques for producing combinatorial libraries of variants. In still other embodiments of the present invention, the nucleotide sequences of the present invention may be engineered in order to alter a maspin coding sequence including, but not limited to, alterations that modify the cloning, processing, localization, secretion, and/or expression of the gene product. For example, mutations may be introduced using techniques that are well known in the art (e.g., site-directed mutagenesis to insert new restriction sites, alter glycosylation patterns, or change codon preference, etc.).

II. Detection of Disorders Associated with Bone Formation

The present invention provides methods of detecting disorders associated with bone formation (e.g., osteoporosis, rheumatoid arthritis, Paget's disease of bone, bone lesions (e.g., cancerous bone lesions), etc.) comprising detecting and quantifying maspin expression. The present invention is not limited to particular methods for detecting disorders associated with bone formation. In some embodiments, the methods comprise obtaining tissue or fluid samples from a subject, quantifying or detecting the amount of maspin expression, and comparing the quantified amount with maspin expression levels established as normal (e.g., based on population average, based on level from subject at previous time point). In some embodiments, subjects having diminished maspin expression (e.g., in comparison with maspin expression levels established as normal) are considered to be at risk for developing and/or having a bone formation disorder. The methods are not limited to particular disorders associated with bone formation. Examples of disorders associated with bone formation include, but are not limited to, osteoporosis, rheumatoid arthritis, Paget's disease of bone, bone lesions (e.g., bone lesions resulting from metastasizing cancer (e.g., breast cancer)). In some embodiments, the disorder is any disorder associated with diminished and/or inhibited osteoblast cell formation.

The methods are not limited to a particular manner of characterizing maspin expression. In some embodiments, maspin expression is characterized (e.g., detected and/or quantified) using microarray (e.g., nucleic acid or tissue microarray), immunohistochemistry, Northern blot analysis. In some embodiments, maspin expression is characterized (e.g., detected and/or quantified) using antibodies (e.g., antibodies directed toward maspin). In some embodiments, detection is performed on cells or tissue after the cells or tissues are removed from the subject. In other embodiments, detection is performed by visualizing maspin in cells and tissues residing within the subject. In some embodiments, detection of maspin is accomplished by measuring the accumulation of corresponding mRNA in a tissue sample. mRNA expression may be measured by any suitable method. In some embodiments, RNA is detected by Northern blot analysis. Northern blot analysis involves the separation of RNA and hybridization of a complementary labeled probe. In some embodiments, RNA (or corresponding cDNA) is detected by hybridization to an oligonucleotide probe. In some embodiments, reverse-transcriptase PCR (RT-PCR) is used to detect the expression of maspin RNA. In some embodiments, detection of maspin is accomplished through detecting and/or quantifying maspin protein levels. Protein expression may be detected by any suitable method. In some embodiments, proteins are detected by binding of an antibody specific for maspin. Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In some embodiments, comparing expression of maspin over a period of time may be used to test the efficacy of a treatment (e.g., drugs directed toward treating disorders associated with bone formatiuon) and/or may be used to test the efficacy of a new form of treatment (e.g., new drugs directed toward treating disorders associated with bone formation).

In some embodiments, detection of the presence or absence (or characterization) of a bone formation disorder is accomplished through comparing expression levels of maspin to established thresholds. For example, in some embodiments, characterizing the present or absence of a bone formation disorder is accomplished through comparing expression levels of maspin with established maspin threshold levels (e.g., established maspin threshold level for low risk for developing a particular bone formation disorder; established maspin threshold level for medium risk for developing a particular bone formation disorder; established maspin threshold level for high risk for developing a particular bone formation disorder). Established threshold levels may be generated from any number of sources, including but not limited to, groups of subjects (of any age and/or gender) having particular disorders associated with bone formation (e.g., osteoporosis, rheumatoid arthritis, Paget's disease of bone, bone lesions (e.g., bone lesion resulting from metastastizing cancer (e.g., breast cancer), etc.). Any number of subjects within a group may be used to generate an established threshold (e.g., 5 subjects, 10 subjects, 20, 30, 50, 500, 5000, 10,000, etc.). Threshold levels may be generated with any type or source of bodily sample from a subject.

### III. Therapeutics

In some embodiments, the present invention provides methods for treating or researching disorders associated with bone formation comprising altering (e.g., increasing) maspin expression and/or activity. In some embodiments, altering (e.g., increasing) maspin expression and/or activity comprises providing to cells (e.g., osteoblast cells) a composition comprising a maspin. In some embodiments, altering (e.g., increasing) maspin activity comprises altering the targets of maspin, altering components of pathways associated with maspin activity, altering genes upregulated or downregulated in response to maspin expression.

The present invention is not limited by the type of agent used to alter (e.g., increase) maspin expression. Indeed, any compound, pharmaceutical, small molecule or agent (e.g., antibody, protein or portion thereof) that can alter (e.g., increase) maspin expression and/or activity is contemplated to be useful in the methods of the present invention. In some embodiments, the agent used to alter (e.g., increase) maspin expression is recombinant maspin. In some embodiments, the agent used to alter (e.g., increase) maspin expression is a maspin expressing cell line (e.g., TM40D tumor cell line (see, e.g., Kittrell F S, et al., Cancer Res 1992;52(7): 1924-32; herein incorporated by reference in its entirety)).

The present invention is not limited to treating or researching a particular disorder associated with bone. In some embodiments, the disorder is osteoporosis. In some embodiments, the disorder is rheumatoid arthritis. In some embodiments, the disorder is Paget's disease of bone. In some embodiments, the disorder is a bone lesion (e.g., bone lesion resulting from metastastizing cancer (e.g., breast cancer).

In some embodiments, the composition comprising maspin is co-administered with an agent useful for treating osteoporosis. In some embodiments, the composition comprising maspin is co-administered with an agent useful for treating rheumatoid arthritis. In some embodiments, the composition comprising maspin is co-administered with an agent useful for treating bone lesions (e.g., bone lesion resulting from metastastizing cancer (e.g., breast cancer)) (e.g., chemotherapeutic agents).

The present invention is not limited to a particular agent for treating osteoporosis. A variety of agents for treating osteoporosis are contemplated to be useful in the present invention including, but not limited to, hormone replacement therapy agents (e.g., estrogen, estradiol, ethinyl estradiol, norethindrone), bisphosphonates (e.g., alendronate, risedronate, etidronate, ibandronate), androgens (e.g., testosterone), parathyroid hormone (e.g., teriparatide), selective estrogen-receptor modulators (e.g., raloxifene), and calcitonin-salmon.

The present invention is not limited to a particular agent for treating rheumatoid arthritis. A variety of agents for treating rheumatoid arthritis are contemplated to be useful in the present invention including, but not limited to, non-steroidal anti-inflammatory drugs (e.g., ibuprofen), cyclooxygenase-2 inhibitors (e.g., celecoxib), disease-modifying agents (e.g., methotrexate, leflunomide, azathioprine, sulfasalazine, infliximab, adalimumab, etanercept), immunomodulators (e.g., abatacept), interleukin-1 receptor inhibitors (e.g., anakinra), and glucocorticoids (e.g., prednisone).

In some embodiments, the present invention provides compositions comprising expression cassettes comprising a nucleic acid encoding maspin, and vectors comprising such expression cassettes. The methods are generally applicable across many species. The therapeutic methods of the invention are optimally achieved by targeting the therapy to the affected cells (e.g., osteoblast cells). Introduction of molecules carrying genetic information into cells is achieved by any of various methods including, but not limited to, directed injection of naked DNA constructs, bombardment with gold particles loaded with the constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. patent application Nos. 6,033,908, 6,019,978, 6,001,557, 5,994,132, 5,994,128, 5,994,106, 5,981,225, 5,885,808, 5,872,154, 5,830,730, and 5,824,544, each of which is incorporated herein by reference in their entireties. In some embodiments, cells are transfected ex vivo and transplanted into a subject.

Vectors may be administered to subject in a variety of ways. For example, in some embodiments of the present invention, vectors are administered into osteoblast cells or tissue using direct injection. In other embodiments, administration is via the blood or lymphatic circulation of a patient a bone formation disorder (e.g., osteoporosis). Exemplary dose levels of adenoviral vector are preferably 10⁸ to 10¹¹ vector particles added to the perfusate.

In some embodiments, the present invention provides compositions and methods for treating and empirically investigating bone lesions (e.g., bone lesions resulting from metastastizing cancer (e.g., breast cancer)). Breast cancer has extremely high morbidity with bone metastasis (see, e.g., Guise T A, et al., Endocr Rev 1998;19(1):18-54; herein incorporated by reference in its entirety). Clinical studies indicate about 70% of patients with breast cancers developed bone tumors, while a smaller number of patients manifested lung or liver metastasis (see, e.g., Mundy G R, Cancer 1997;80(8 Suppl):1546-56; Fedarko N S, et al., Clin Cancer Res 2001;7(12):4060-6; each herein incorporated by reference in their entireties). Several molecules that control bone tumor growth and bone homeostasis have been recently identified. For example, breast cancer cells produce PTHrP (parathyroid hormone-related protein), which causes bone destruction (see, e.g., Guise T A, J Clin Invest 1996;98(7):1544-9; herein incorporated by reference in its entirety). RANKL (receptor activator of nuclear factor κB ligand), a TNF ligand transmembrane protein, is an osteoclast differentiation factor critical to osteoclast recruitment (see, e.g., Yasuda H, et al., Proc Natl Acad Sci USA 1998;95(7):3597-602; Hsu H, et al., Proc Natl Acad Sci USA 1999;96(7):3540-5; Kodaira K, Gene 1999;230(1):121-7; each herein incorporated by reference in their entireties). RANKL exerts its effects through its cognate receptor, RANK, which is expressed on osteoclast precursor cells (see, e.g., Nakagawa N, Biochem Biophys Res Commun 1998;253(2):395-400; herein incorporated by reference in its entirety). RANKL-deficient mice developed severe osteopetrosis and a tooth eruption defect as a result of an inability of osteoblasts to support osteoclastogenesis (see, e.g., Nakagawa N, Biochem Biophys Res Commun 1998;253(2):395-400; herein incorporated by reference in its entirety).

Tumor invasion and metastasis is a complicated process involving local invasion, intravasation and extravasation into and out of blood vessels, and tumor growth at the secondary sites. Maspin is a unique member of the serpin family (see, e.g., Zou Z, et al., Science 1994; 263(5146):526-9; herein incorporated by reference in its entirety) whose down regulation is associated with the development of breast cancer (see, e.g., Sheng S, J Biol Chem 1994;269(49):30988-93; Sager R, et al., Cold Spring Harb Symp Quant Biol 1994;59:537-46; Zhang M, et al., Proc Natl Acad Sci USA 1997; 94(11):5673-8; Seftor R E, Cancer Res 1998;58(24):5681-5; Hendrix M J; Nat Med 2000;6(4):374-6; Pemberton P A, J Biol Chem 1995;270(26): 15832-7; each herein incorporated by reference in their entireties). Previous experiments demonstrated that recombinant maspin made in bacteria, yeast, or insect, inhibits invasion and motility of mammary carcinoma cells in culture (see, e.g., Zou Z, et al., Science 1994; 263(5146):526-9 (9); Sheng S, et al., Proc Natl Acad Sci USA 1996;93(21): 11669-74; Zhang M, Mol Med 1997;3(1):49-59; Sternlicht M D, Clin Cancer Res 1997;3(11):1949-58; Pemberton P A, J Histochem Cytochem 1997; 45(12):1697-706; Sager R, Curr Top Microbiol Immunol 1996;213((Pt 1)):51-64; each herein incorporated by reference in their entireties). Maspin also inhibits endothelial cell motility and angiogenesis in a xenograft tumor model (see, e.g., Zhang M, et al., Nat Med 2000;6(2):196-9; herein incorporated by reference in its entirety), suggesting, for example, that the anti-angiogenesis property of maspin is related to its tumor suppressor activity. In addition, maspin has been shown to inhibit TM40D tumor metastasis to lung and intestine (see, e.g., Shi H Y, Cancer Res 2001;61(18):6945-51; herein incorporated by reference in its entirety), and maspin-expressing prostate tumors exhibited reduced angiogenesis in a xenograft prostate tumor model (see, e.g., Sheng S, et al., Proc Natl Acad Sci USA 1996;93(21):11669-74; herein incorporated by reference in its entirety).

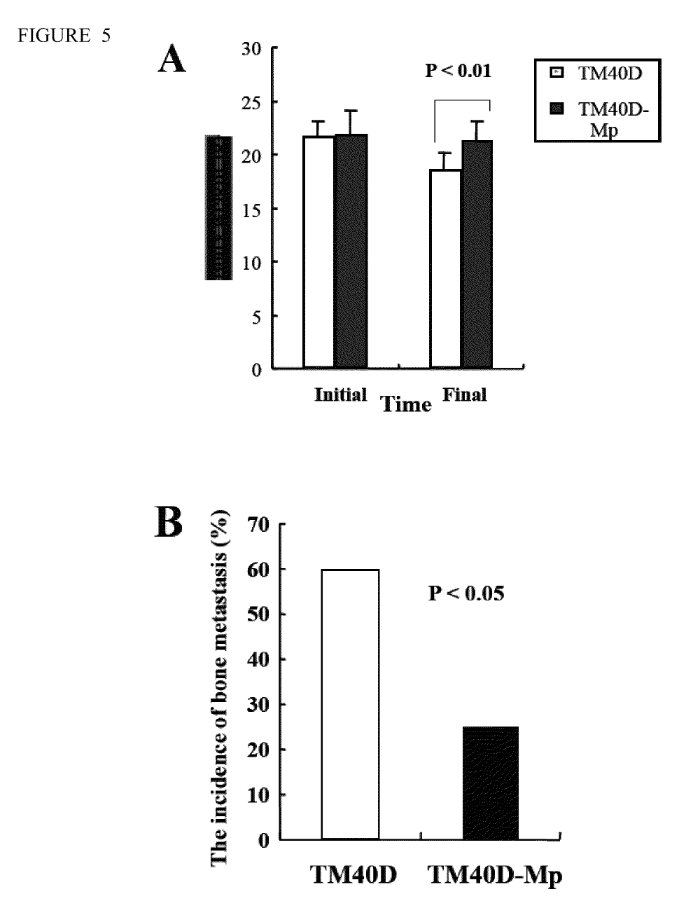

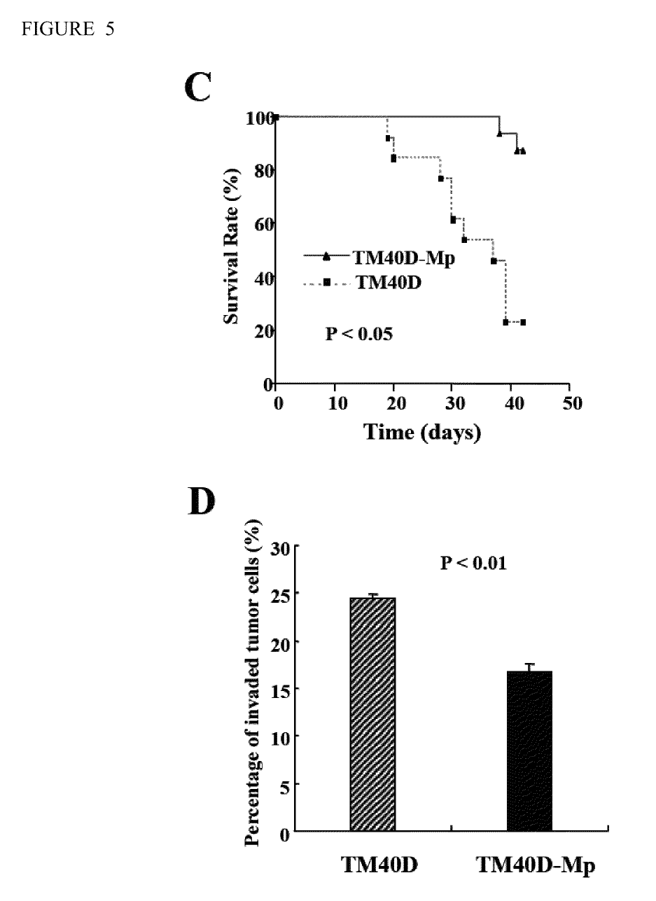

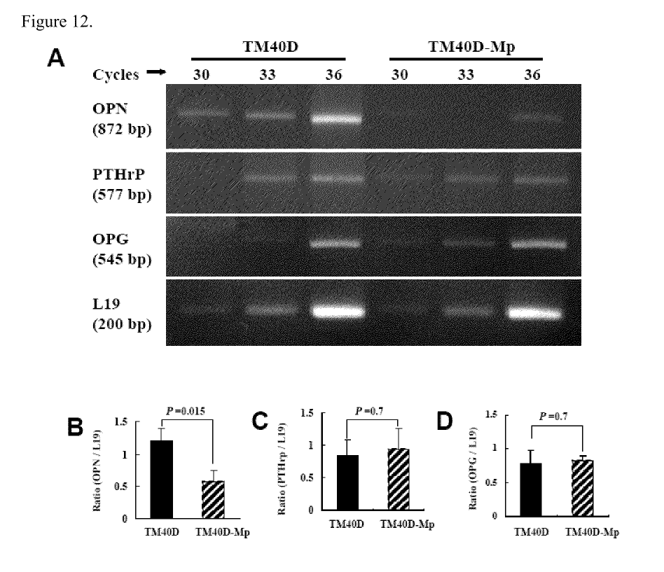

In experiments conducted during the course of developing embodiments for the present invention, it was shown that TM40D-Mp tumor cells exhibit a reduced rate of bone metastasis (FIG. 5). Transendothelial cell migration assay showed that maspin inhibits tumor cell invasion through the endothelial cell layer. RT-PCR analysis showed that maspin-expressing tumor cells exhibited a reduced level of OPN expression compared to that of TM40D tumor cells (FIG. 12B). OPN has been reported to be secreted from tumor cells to induce angiogenesis in the bone tumor environment (see, e.g., Hirama M, et al., Cancer Lett 2003;198(1):107-17; Takahashi F, et al., Int J Cancer 2002;98(5):707-12; each herein incorporated by reference in their entireties). While not limited to a particular mechanism, these results suggest that maspin inhibits tumor angiogenesis in the bone by regulating the secretion of angiogenic factors from tumor cells.

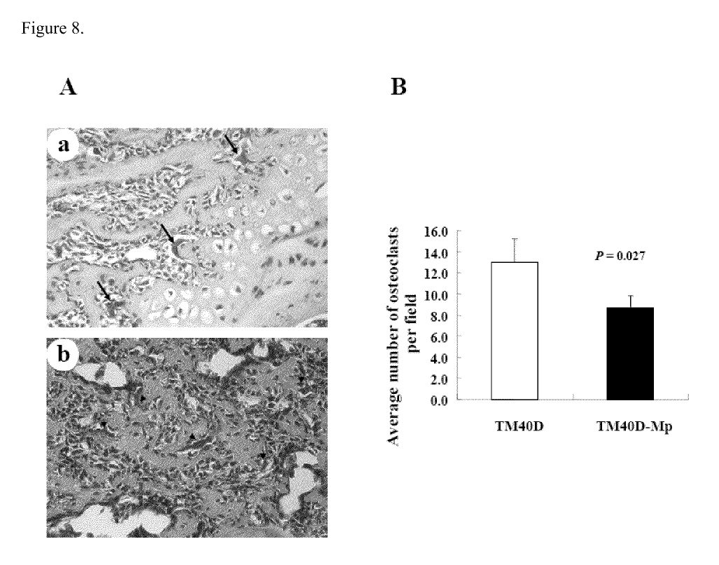

In experiments conducted during the course of developing embodiments for the present invention, it was shown that maspin modulates the pattern of breast cancer bone metastasis. In particular, it was shown that inoculation of the TM40D tumor strain to the mice produces exclusively the osteolytic bone lesion. However, maspin expressing TM40D-Mp tumors produce both osteoclastic and osteosclerotic lesions. In vivo and in vitro experiments revealed that maspin-expressing tumors cause a reduction in osteoclast differentiation and an increase in osteoblast differentiation (FIG. 8, 9, and 11), suggesting, for example, that the presence of maspin modulates the tumor-bone host interaction. It is noted that in most cases of breast cancer bone metastasis, tumor cells do not directly cause bone resorption or induce new bone formation, rather the tumor cells alter bone homeostasis by influencing the functions of osteoblasts and osteoclasts (see, e.g., Traianedes K, et al., Endocrinology 1998; 139(7):3178-84; Chikatsu N, et al., Biochem Biophys Res Commun 2000;267(2):632-7; each herein incorporated by reference in their entireties). In addition, osteoblastic cells co-cultured with TM40D-Mp cells were shown to have a reduced level of RANKL expression and increased cyclin D1 expression compared to that co-cultured with TM40D cells.

Accordingly, in some embodiments, the present invention provides compositions and methods for preventing and/or treating bone lesions (e.g., bone lesions resulting from metastasizing breast cancer) in a subject through administration of maspin (e.g., a therapeutically effective amount of maspin). In some embodiments, the administered mapsin results in decreased RANKL expression. In some embodiments, the administered maspin results in increased cyclin D1 expression. In some embodiments, the administered maspin results in increased osteoblast cell proliferation. In some embodiments, the compositions and/or methods are co-administered with an anti-cancer agent. The compositions and/or methods are not limited to a particular type or kind of anti-cancer agent, nor is it limited to the administration of a particular number of anti-cancer agents. In some embodiments, the anti-cancer agent is select from at least one of the group consisting of Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); 2-chlorodeoxyadenosine (2-Cda), Antiproliferative agents, Piritrexim Isothionate, Antiprostatic hypertrophy agents, Sitogluside, Benign prostatic hypertrophy therapy agents, Tamsulosin Hydrochloride, Prostate growth inhibitor agents, Pentomone, and Radioactive agents, Fibrinogen 1 125; Fludeoxyglucose F 18; Fluorodopa F 18; Insulin I 125; Insulin I 131; lobenguane I 123; lodipamide Sodium I 131; lodoantipyrine I 131; lodocholesterol I 131; lodohippurate Sodium I 123; lodohippurate Sodium I 125; lodohippurate Sodium I 131; lodopyracet I 125; lodopyracet I 131; lofetamine Hydrochloride I 123; lomethin I 125; lomethin I 131; lothalamate Sodium I 125; lothalamate Sodium I 131; lotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; and Triolein I 131.

### IV. Pharmaceutical Compositions

The present invention further provides pharmaceutical compositions (e.g., comprising agents designed to increase maspin expression and/or activity). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing may be dependent on severity and responsiveness of the disease state (e.g., stage of bone formation disorder) to be treated, with the course of treatment lasting from several minutes, hours, and/or days to several months. Optimal dosing schedules can be calculated from measurements of drug accumulation in the body of the subject (e.g., patient). The administering physician can easily determine optimum dosages, dosing methodologies and repetition rates. Optimum dosages may vary depending on the relative potency of individual agents to be administered, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence/occurrence of the disease state, wherein the treatment is administered in maintenance doses, ranging from 0.01 μg to 100 g per kg of body weight, once or more daily, to once every, for example, hour, day, week and/or month.

### V. Drug Screening

In some embodiments, the present invention provides drug screening assays (e.g., to screen for new drugs for treating disorders associated with bone formation). For example, in some embodiments, the present invention provides methods of screening for compounds that alter (e.g., increase), directly or indirectly, the presence of maspin expression and/or activity.

In one screening method, candidate compounds are evaluated for their ability to alter maspin activity and/or expression by contacting a compound with a cell (e.g., an osteoblast cell) and then assaying for the effect of the candidate compounds on the presence or expression of maspin. In some embodiments, the effect of candidate compounds on expression or presence of maspin is assayed for by detecting the level of maspin present within the sample. In other embodiments, the effect of candidate compounds on expression or presence of maspin is assayed for by detecting the level of maspin present in the extracellular matrix.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell etal., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Libraries of compounds may be presented in solution (e.g., Houghten, Biotechniques 13:412-421 (1992)), or on beads (Lam, Nature 354:82-84 (1991)), chips (Fodor, Nature 364:555-556 (1993)), bacteria or spores (U.S. Pat. No. 5,223,409; herein incorporated by reference), plasmids (Cull et al., Proc. Nad. Acad. Sci. USA 89:18651869 (1992)) or on phage (Scott and Smith, Science 249:386-390 (1990); Devlin Science 249:404-406 (1990); Cwirla et al., Proc. Natl. Acad. Sci. 87:6378-6382 (1990); Felici, J. Mol. Biol. 222:301 (1991)).

This invention further pertains to novel agents identified by the above-described screening assays. Accordingly, it is within the scope of this invention to further use an agent identified as described herein (e.g., a maspin modulating agent) in an appropriate animal model (such as those described herein) to determine the efficacy, toxicity, side effects, or mechanism of action, of treatment with such an agent. Furthermore, novel agents identified by the above- described screening assays can be, e.g., used for treatments as described herein.

### VI. Kits

In yet other embodiments, the present invention provides kits for the detection, characterization, and/or treatment of disorders associated with bone formation (e.g., osteoporosis). In some embodiments, the kits contain antibodies specific for maspin polypeptides. In some embodiments, the kits further contain detection reagents and buffers. In other embodiments, the kits contain reagents specific for the detection of maspin nucleic acids. In preferred embodiments, the kits contain all of the components necessary and/or sufficient to perform a detection assay, including all controls, directions for performing assays, and any necessary software for analysis and presentation of results.

### Experimental

The following examples are provided in order to demonstrate and further illustrate certain preferred embodiments and aspects of the present invention and are not to be construed as limiting the scope thereof.

### EXAMPLE I

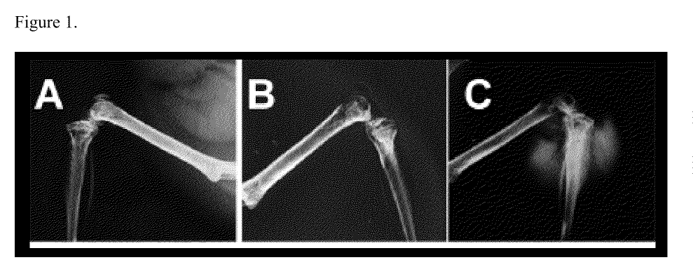

This example demonstrates that maspin promotes new bone formation in a mouse model. Representative radiographs for bone lesions from mice were inoculated by intracardiac injection of mouse mammary TM40D (control) and maspin-overexpression TM40D-Mp tumor cells. As shown in FIG. 1A, a typical image of the long bone from a normal control mouse is presented. FIG. 1B shows a representative image of bone lesions from mice inoculated with TM40D tumor cells. FIG. 1C shows representative image of bone lesions from mice inoculated with TM40D-Mp cells.

### EXAMPLE II

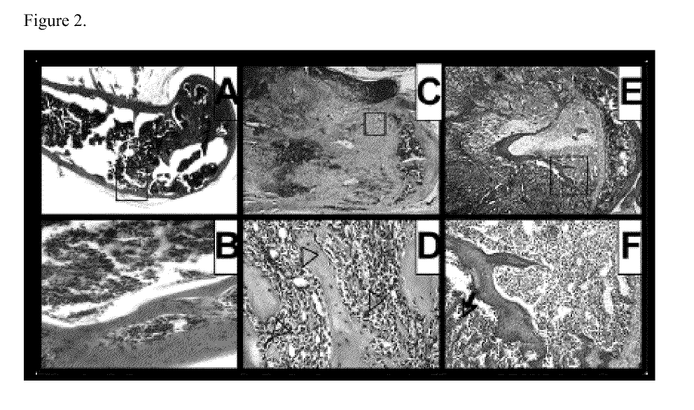

This example describes histological analysis of bone lesions from mice inoculated with TM40D and TM40D-Mp tumor cells. FIG. 2, panels A-B, present typical H&E sections of the long bone of normal mouse. FIG. 2, panels C-D, show representative section of bone lesions from mice inoculated with TM40D tumor cells wherein the arrow indicates the area of tumor cells. FIG. 2, panels E-F, show representative section of bone lesions from mice inoculated with TM40D-Mp cells. As shown in FIG. 2, significant new bone formation was observed with overexpression of maspin.

### EXAMPLE III

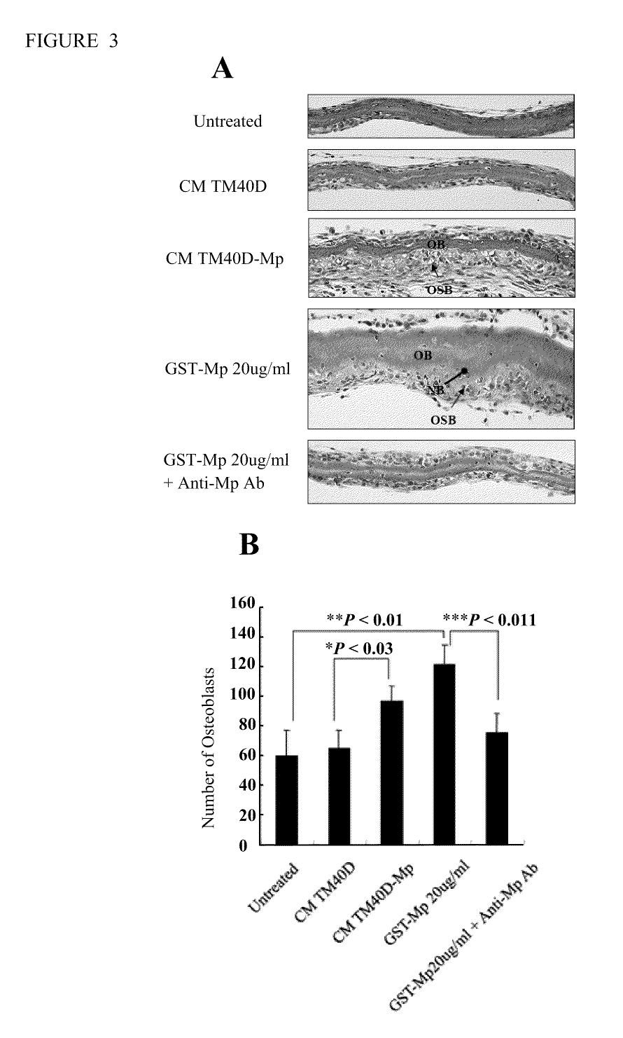

This example describes the effect of maspin on osteoblast proliferation and new bone formation in an organ culture model. As shown in FIG. 3A, calvariae were removed from 4 days-old BABL/c new-born mice. The calvariae were either treated with or without concentrated conditional medium from TM40D and maspin-overexpression TM40D-Mp cells or treated with recombinant GST-Mp or GST-Mp with or without an anti-maspin antibody for 7 days. As shown, osteoblast proliferation and new bone formation was demonstrated. FIG. 3B shows quantitation of the average number of osteoblasts under the different treatments shown in FIG. 3A. Three randomly selected fields at the same pixel size were viewed under the microscope (X200), and the number of defined osteoblasts was counted. The average number of osteoblasts was analyzed using a statistical program. *P=0.03; **P<0.01; ***P=0.011.

### EXAMPLE IV

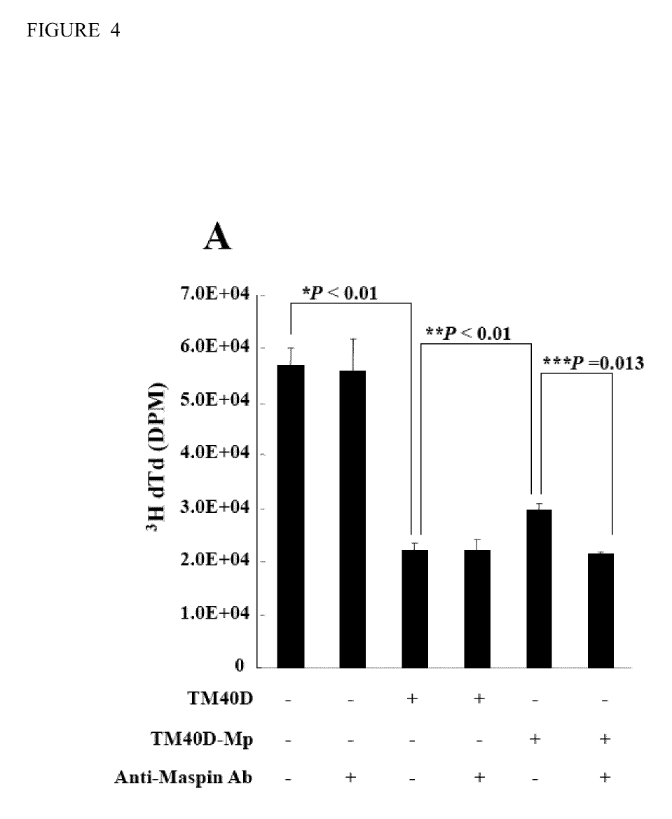

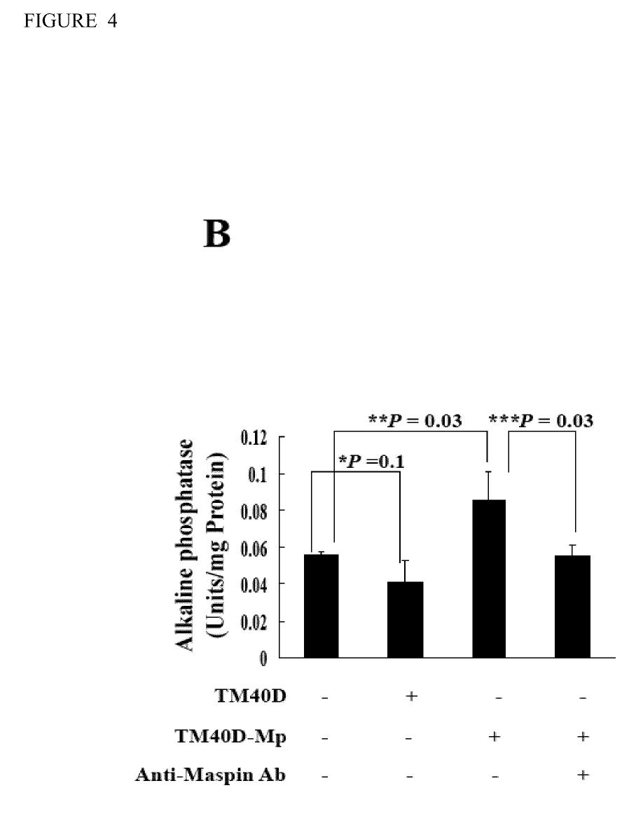

This example describes the effects of maspin on osteoblats proliferation and differentiation. As shown in FIG. 4A, osteoblast cells were co-cultured with TM40D and TM40D-Mp cells for 48 hours. DNA synthesis in osteoblasts was assessed by 3H-Thymidine incorporation. Data were obtained from triplicate experiments. Values are the mean±SD. * P<0.01; **P<0.01; ***P<0.05. As shown in FIG. 4B, the effect of maspin on osteoblastic cell differentiation was analyzed by alkaline phosphatase activity assay. Osteoblastic cells treated with TM40D-Mp cells had significantly increased alkaline phosphatase activity compared to that were treated with TM40D cells.

### EXAMPLE V

This example describes the materials and methods used in Examples VI through XIII.

Cells and Cell Culture

TM40D mammary tumor cell line was established from a TM40D primary tumor that arose in serially transplanted TM40D pre-neoplastic outgrowth line (see, e.g., Kittrell F S, et al., Cancer Res 1992;52(7): 1924-32; herein incorporated by reference in its entirety). Maspin-expressing TM40D tumor cell line (TM40D-Mp) was derived from stable transfectants carrying a retroviral maspin expression construct (see, e.g., Shi H Y, et al., Cancer Res 2001;61(18):6945-51; herein incorporated by reference in its entirety). Both TM40D and TM40D-Mp cells were grown in Dulbecco's Modified Eagle Medium/F 12 (DMEM/F12) medium supplemented with 2% fetal bovine serum (FBS), epidermal growth factor, and insulin (see, e.g., Kittrell FS, et al., Cancer Res 1992;52(7):1924-32; herein incorporated by reference in its entirety). Human umbilical vein endothelial cells (HUVECs) (Clonetics, San Diego, Calif.) were cultured in endothelial basal medium (EBM) supplemented with 2% FBS, 12 mg/ml bovine brain extract (BBE), 10 ng/ml human epidermal growth factor (hEGF), 1 mg/ml hydrocortisone, 50 mg/ml gentamicin, and 50 ng/ml amphotericin (Clonetics, CA). MC3T3-E1, an osteoblast precursor cell line, was obtained from ATCC and maintained in DMEM (Gibco/BRL Life Technologies, NY) supplemented with 10 mg/L ribonucleosides and deoxyribonucleosides, 10% FBS, 100 U/ml penicillin, and 100 mg/ml streptomycin.

Intracardiac Injection of Tumor Cells in Balb/c Mice

Female 4-week-old BALB/c mice were purchased from Harlan Sprague Dawley, Inc. (Indianapolis, Ind.), and housed in a pathogen-free environment. The intracardiac injection was modified based on a protocol described by Li et al. (see, e.g., Li Y, Cancer Res 2001;61(18):6906-11; herein incorporated by reference in its entirety). Briefly, subconfluent TM40D and TM40D-Mp cell lines were fed with DMEM/F12 containing 2% FBS 24 h before they were harvested. Cells were harvested with 0.05% trypsin, washed with PBS, and resuspended at a density of 2.5×10⁶ cells/ml. Before injection, animals were anesthetized with ketamine (30 μg/g) and xylazine (1.5 μg/g). Cells (5×10⁵) were slowly injected into the left cardiac ventricles of animals using a 27-gauge needle. A successful injection was characterized visually by the pumping of arterial blood into the syringe. After injection, animals were placed on a heating pad to recover from the anesthesia. The body weight of each animal was measured twice a week right after mouse was inoculated with tumor cells. The development of bone metastases was monitored weekly by X-ray radiography starting from three weeks after the tumor injection.

Determination of Bone Metastases by X-Ray Radiography

Animals were anesthetized and placed on a transparent board in prone and lateral positions. The board was placed against an X-ray film (22×27 mm; X-OMAT AR; Kodak, Rochester, N.Y.), and animals were exposed to X-ray at 20 kV for 15 s in a Faxitron® radiographic inspection unit (model 43855A). Exposed films were developed using an automatic film processor (Kodak RP X-OMAT). Radiographs of bone were evaluated for the presence of bone lesions.

Histological Analysis

Histological analysis was performed based on a method described in Li et al. (see, e.g., Li Y, Cancer Res 2001;61(18):6906-11; herein incorporated by reference in its entirety). Animals were sacrificed at week 6 after the inoculation of tumor cells, the front and hind limbs were excised for the removal of muscle tissues, and were fixed and decalcified in Cal-Ex II solution (Fisher Scientific, Fairlawn, N.J.) for 24 h. The tissues of lung, liver, and intestine also were excised and fixed with 10% formalin. All tissues were embedded in paraffin and sectioned at 5 μm. Histological sections were stained with hematoxylin and eosin (H&E) for histological analysis. Micrographs were taken using a SPOT digital camera under a Leica DM IRB microscope, and images were processed using Adobe® Photoshop® software.

Osteoclast Formation Analysis Using Tartrated-resistant Acid Phosphatase (TRAP) Staining

Staining of bone tissue for detection of tartrate-resistant acid phosphatase was performed as follows. Serial sections (5 μm in thickness) of femurs from the mice with radiographically verified lesions (both osteolytic and osteoblastic) were used for TRAP staining, using a commercial kit (Sigma, St. Louis, Mo.) according to the manufacturer's instructions. After the TRAP staining, osteoclasts were identified as TRAP-positive multinucleated cells and appeared in red color. Five fields at the same pixel size were randomly selected for each slide, and the number of osteoclasts was manually counted. The numbers of osteoclasts in each section were calculated and analyzed by a statistical program (t-Test).

Preparation of Mouse Bone Marrow Cells

Mouse bone marrow cells were obtained from 5-week-old female BALB/c mice as described by Michigami et al. (see, e.g., Michigami T, et al., Blood 2000;96(5):1953-60; herein incorporated by reference in its entirety). Femurs and tibiae were dissected aseptically by cutting off both bone ends and flushing out the cells from bone marrow cavity. Collected cells were incubated in 100 mm culture dishes in the DMEM medium described above for 2 hours. Non-adherent cells containing hemopoietic osteoclast precursors and stromal cells were harvested for co-culture with the tumor cells.

Co-Culture of Bone Marrow Cells with Breast Tumor Cells and Osteoclast Differentiation Assay

Co-culture of bone marrow cells was based on a method described in Michgami et al. (see, e.g., Michigami T, et al., Blood 2000;96(5):1953-60; herein incorporated by reference in its entirety) and modified as follows. Briefly, bone marrow cells (1×10⁶/well) were plated onto 24-well culture plates. After cells were adhered onto the surface of plates, TM40D cells or TM40D-Mp cells (1×10³/well) were inoculated with the bone marrow cells. On day 1, the cells were co-cultured in 300 μL of culture medium, on day 2, 300 μL of fresh culture medium was gently added to each well, and on day 4,300 μL of the medium was replaced with the same volume of fresh medium. On day 6, the cells were fixed with 10% formalin in PBS for 10 min and TRAP staining was performed using a commercial kit (Sigma) according to the manufacturer's instructions (Sigma, Mo.). TRAP(+) MNC containing 2 or more nuclei were defined as osteoclasts. The number of osteoclasts was manually counted from five randomly selected fields. The experiment was repeated three times and data were analyzed using a statistical program (t-Test).

Primary Cultures of Mouse Calvaria Osteoblasts

Cultures of PMOs (primary mouse osteoblasts) were obtained from the calvaria as described by Yang et al. (see, e.g., Yang J, et al., Cancer Res 2001;61(14):5652-9; herein incorporated by reference in its entirety). Briefly, calvaria were taken from the day 4 BALB/c mice and were digested for 15 min in a shaking incubator at 37° C. with 15 ml of α-MEM containing 0.1 mg/ml collagenase P (Boehringer Mannheim, Corp, Indianapolis, Ind.), 2.5% trypsin/EDTA (Life Technologies, Inc.), streptomycin, and penicillin. The mixtures were gently shaken by hand for 20 s every 5 min during the procedure. The digestion medium and released cells were then discarded. After two rounds of digestion, cell suspensions were transferred to new tube and washed with α-MEM plus 10% FBS. This procedure was repeated three times, cells were plated with α-MEM plus 10% FBS for 48 h. Cultured cells were passed for additional experiments.

Co-Culture of PMOs with Mouse Tumor Cells

PMOs were seeded in tissue culture plates (5,000 cells/cm²), whereas TM40D and TM40D-Mp cells were seeded in cell-culture inserts (1,000 cells/cm²) (0.4-μm pore; Falcon/Becton Dickinson Labware, Franklin Lakes, N.J.). After 24 hr of cell culture, the inserts with tumor cells were placed into he plates with PMOs in co-culture medium containing α-MEM plus 5% Dulbecco's Modified Eagle Medium. After 48h of co-culture, PMOs were assayed for proliferation index using the method of [³H]-Thymidine incorporation (NEN Life Science Products, Boston, Mass.).

Alkaline Phosphate Activity

PMOs were co-cultured with tumor cells as described above. After 4 days of co-culture, the inserts containing the tumor cells were removed, and PMOs were maintained in culture until they reach confluence. PMOs were then placed in differentiation medium (α-MEM plus 10% FBS, 100 μg/ml ascorbic acid, and 10 nM sodium β-glycerophosphate) in the presence of BMP-2 (20 ng/ml). At day 4, cultured medium was replaced with the differentiation medium, and the old culture media were harvested and tested for alkaline phosphatase activity. Alkaline phosphatase activity was determined using a kit from Sigma, Inc.

Preparation of Conditional Medium from TM40D and TM40D-Mp Cells

The procedure was based on a method described in Thomas et al. (see, e.g., Thomas R J, et al., Endocrinology 1999; 140(10):4451-8; herein incorporated by reference in its entirety), and modified as follows. Briefly, TM40D and TM40D-Mp cells were grown to 80-90% confluence in DMEM/F12 media containing 2% FBS. The media were removed and the cells were washed with phosphate-buffered saline. Cells were incubated for 24 hr in serum-free media. After 2 hr, conditioned media were collected and protease inhibitors were added [10 nM leupeptin, 0.8 nM aprotinin, 28 mM phenylmethyl-sulfonyl fluoride (Sigma)]. Conditioned media were concentrated with a Biomax centrifugal filter device (MW cutoff 30 kD, Millipore Corp., Bedford, Mass.), and then frozen and lyophilized. Before use, lyophilized media were rehydrated in water and protein concentration was determined using a Bio-Rad DC protein assay kit (Bio-Rad Laboratories, Hercules, Calif.).

Mouse Calvarial Organ Culture

Mouse calvarial organ culture was performed as described previously (see, e.g., Traianedes K, et al., Endocrinology 1998;139(7):3178-84; herein incorporated by reference in its entirety). Briefly, calvarial bones were excised from 4-day-old pups of BABL/c mice and cut into half along the sagittal suture. Each half of the calvaria was placed on a stainless steel grid in a 12-well tissue culture dish. Tissues in each well were cultured in 1 ml of Biggers-Gwatokin-Jackson medium (Sigma Chemical Co.) containing 0.1% BSA, and were supplemented with 20 mg/ml conditioned medium (CM) harvested from the TM40D and TM40D-Mp cell culture (v/v) or with GST-Mp recombinant protein at 20 μg/ml in the absence or presence of an anti-maspin antibody. The culture medium was changed at day 3, and cells were cultured until day 7. At the end of the cultures, the calvaria halves were fixed, decalcified, paraffin-embedded, sectioned, stained with hematoxylin and eosin, and photographed with a high-resolution video camera (Sony 3CCD) linked to a Nikon microscope. Representative images were captured using the Osteometrics software program at X 200 magnification. The number of osteoblasts on the bone surfaces were determined by histomorphometric analysis using the Osteomeasure System (Osteometrics Inc., Atlanta, Ga.). The number of osteoblasts on both the endosteal and periosteal surfaces of the calvaria was counted automatically by using the software.

Treatment of MC3T3-E1 Cells with Tumor Cell Conditional Medium

Osteoblastic MC3T3-EL cells were plated in 24-well plates at 50,000 cells/cm² and incubated overnight in 10% FBS-containing media. The following day, designated as day 0, the media were removed and replaced with 10% FBS-containing media plus 25 mg/ml CM and media were changed every 48 hr. The cells were harvested and subjected to RNA extraction on day 5.

Semi-Quantitative Reverse Transcriptase-PCR

Total RNA was isolated from cells using a single-step RNA isolation method using the TRIZOL® reagent (Gibco/BRL). The primers for osteopontin (OPN) were: sense primer (5′-AGTCGACATGAGATTGGCAGTGATT TGC-3′) (SEQ ID NO: 1) and anti-sense primer (5′-ACTCGAGGCCTCTTCTTTAGTTGACCTC-3′) (SEQ ID NO:2). The primers for PTHrP were: sense primer (5′-GAGCGCAGATGGATCCTAA-3′) (SEQ ID NO:3) and anti-sense primer (5′-GAACTTGGGATTTTGATGCTG-3′) (SEQ ID NO:4). The primers for osteoprotegerin (OPG) were: sense primer (5′-ACCAAAGTGAATGCCGAG-3′) (SEQ ID NO:5) and anti-sense primer (5′-AAGAAACAGCCCAGTGACC-3′) (SEQ ID NO:6). The primers for RANKL were: sense primer (5′-TCCTAACAGAATATCAGAAGACA-3′) (SEQ ID NO:7) and anti-sense primer (5′-AGGCTTGCCTCGCTGGGCCACATC-3′) (SEQ ID NO:8). The primers for Cyclin D1 were: sense primer (5′-TCTACACTGACAACTCTATCCG -3′) (SEQ ID NO:9) and anti-sense primer (5′-TAGCAGGAGAGGAAGTTGTTGG -3′) (SEQ ID NO:10). As an internal control, RT-PCR analyses were carried out using L 19 ribosomal gene primers. Primers for L 19 were as follows: sense primer (5 ′-CTGAAGGTCAAAGGGAATGTG-3′) (SEQ ID NO:11) and anti-sense primer (5-GGACAGAGTCTTGATGAT CTC-3′) (SEQ ID NO:12). The cycling conditions for PCR were as follows: denaturation at 94° C. for 45 s, annealing at 62° C. (for OPN and L19) or 55° C. (for PTHrP, OPG, Cyclin D1 and RANKL) for 45 s, and extension at 72° C. for 2 min. The cycles were repeated 30, 33, or 36 times for the detection of expression levels of OPN, PTHrP and OPG, and 29, 32, and 35 times for the detection of RANKL expression levels. The amplified PCR products were loaded on a 2% agarose gel, the size, in base pairs (bp), of target DNA bands were: 872 bp for OPN, 577 bp for PTHrP, 545 bp for OPG, 249 bp for RANKL, 340 bp for Cyclin D1 and 200 bp for L19. The bands were quantified using Quantity One®-4.2.3 Software (Bio-Rad). The expression of each target gene was determined based on the PCR products produced from each of the three different numbers of cycle repetitions.

Transendothelial Invasion Assay

Transendothelial invasion by TM40D and TM40D-Mp cells was analyzed using a method described by Li et al. (see, e.g., Li Y, et al., Cancer Res 2001;61(18):6906-11; herein incorporated by reference in its entirety) and modified as follows. Briefly, HUVECs (1×10⁵) were plated on a fibronectin-coated polycarbonate membrane insert with 8.0 μm pores in a chemotaxis chamber (Neuro Probe, Inc., Gaithersburg, Md.) for 2 hr. Subsequently, TM40D and TM40D-Mp cells were trypsinized and resuspended in DMEM/F12 containing 2% FBS. The TM40D and TM40D-Mp cells were labeled with ³⁵S-labeled methionine (30 μCi/ml; Amersham Biosciences, Piscataway, N.J.) at 37° C. in a CO₂ incubator for 1 hr. The cell labeling efficiency was evaluated using a liquid scintillation counter. The labeled cells (1×10⁴) were seeded on endothelial cell monolayers and incubated for 20 hr at 37° C. in a CO₂ incubator. The cells that migrated to the bottom of the lower chamber were harvested and subjected to quantification using a liquid scintillation counter. The percentage of invasive cells was calculated by dividing the CPM volume of cells that migrated to the bottom of the lower chamber by the CPM volume of the initially plated cells. The data were analyzed by a statistical analysis (t-Test) using data from triplicate wells.

Statistical Analysis

Incidence of metastasis was analyzed by Chi square statistical method. Survive curves were drawn by the Kaplan-Meier method and analyzed using the log rank test. Differences between groups were compared using the Student's t-test (two-sided analysis). All data shown are given as the mean±SEM. A. P-value of <0.05 was considered significant.

### EXAMPLE VI

This example shows that intracardiac inoculation of mouse mammary TM40D tumor cells induces osteolytic lesions in BALB/c mice. To test whether mouse mammary TM40D tumor cells can induce bone metastasis lesions, BALB/c mice were inoculated with TM40D cells through left cardiac ventricle injection using the method described previously (see, e.g., Li Y, et al., Cancer Res 2001;61(18):6906-11; herein incorporated by reference in its entirety). About 4 weeks following tumor inoculation, many tumor cells-injected mice developed the sign of lameness. These mice were first screened by X-ray radiography. Bone lesions were primarily observed in hind legs with a few mice developed lesion in front legs, as analyzed by X-ray radiography (FIG. 5). To further confirm that the lesions induced by TM40D tumors were osteoclastic in nature, some bone tissues were harvested from mice with positively identified lesions (by X-radiography) for histological analysis. Bone samples were decalcified, embedded, and serially sectioned. As shown in FIG. 5D, tumor cells were indeed present within the bone in a manner typical for osteolytic lesions, and in some cases they occupied a large percent of the space within bone (FIG. 5E).

### EXAMPLE VII

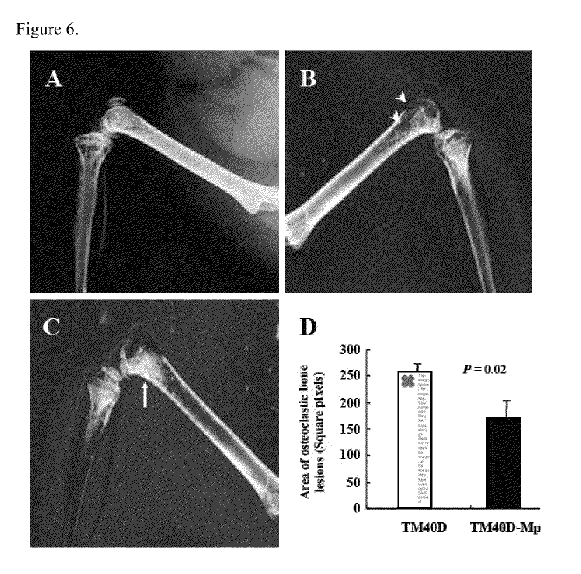

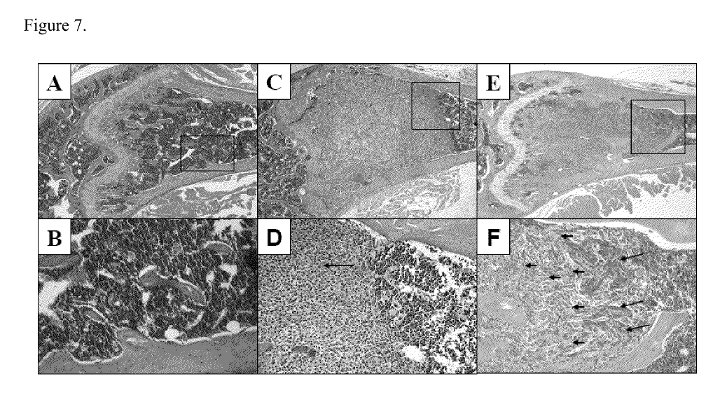

This example shows that maspin inhibits mammary tumor-induced bone lesions. The effect of maspin on mammary tumor bone metastasis was examined. Maspin gene was overexpressed in TM40D cells and established stable expression clones (TM40D-Mp). Equal amount of TM40D or TM40D-Mp tumor cells (5×10⁵) were inoculated into two groups of BABL/c mice. These two groups of mice (15 TM40D and 16 TM40D-Mp) were monitored biweekly for their body weights, starting from one week after tumor inoculation. There was no difference in body weight between TM40D and TM40D-Mp injected mice during the first three weeks. However, the TM40D group began to lose bodyweight after four weeks and developed severe signs of lameness. TM40D-Mp tumor injected mice retained the normal body weight. At the end point of experiment (6 weeks after tumor injection), there was a significant difference in body weight between TM40D and TM40D-Mp groups (18.3 g vs 21.8 g, p<0.01). A significant difference in the rate of bone metastasis was detected between these two groups of mice. Intracardiac injection of TM40D cells resulted in the development of bone lesions in 60% ( 9/15) of the mice. However, only 25% ( 4/16) of the TM40D-Mp-inoculated mice developed bone lesions (p<0.05) (FIG. 6A). The radiographs of all mice from both groups with bone lesions in hind and front legs were analyzed. The number of osteolytic bone lesions was significantly reduced in mice injected with TM40D-Mp tumor cells compared to that from TM40D group (FIG. 7).

About 4 weeks following tumor inoculation, tumor injected mice developed signs of illness, including lameness. Mice were first screened by X-ray radiography. Mice were sacrificed and bone samples were decalcified, embedded, and serially sectioned for histological analysis. Although there was no difference in body weight between TM40D and TM40D-Mp injected mice at initial point of the experiment, the TM40D group mice lost more bodyweights compared to those TM40D-Mp group mice (p<0.01) at the endpoint of the experiment (FIG. 5A). A significant difference was also observed in the rate of bone metastasis in these two groups of mice. Intracardiac injection of TM40D cells resulted in the development of bone lesions in 60% ( 9/15) of the mice. However, only 25% ( 4/16) of the TM40D-Mp cells inoculated mice developed bone lesions (p<0.05) (FIG. 5B). As shown in FIG. 5C, mice that were inoculated with TM40D cells had a higher mortality rate compared to those mice that received TM40D-Mp tumor cells (P<0.05).

It was shown that presence of maspin in TM40D-Mp cells had an inhibitory effect on the incidence of bone metastasis, compared to the control TM40D tumors that do not express maspin (FIG. 5B). While not limited to a particular mechanism, this result was due to reduced invasiveness of TM40D-Mp cells during the extravasation. A transendothelial invasion assay was next performed. As shown in FIG. 5D, the presence of maspin in TM40D-Mp cells significantly reduced the ability of tumor cells to migrate through the endothelial cells (P<0.01).

Although there was no difference in body weight between TM40D and TM40D-Mp injected mice at initial point of the experiment, the TM40D group mice lost more bodyweights compared to those TM40D-Mp group mice (p<0.01) at the endpoint of the experiment (FIG. 5A). A difference was also observed in the rate of bone metastasis in these two groups of mice. Analyses of X-ray radiographs showed that intracardiac injection of TM40D cells resulted in the development of bone lesions in 60% ( 9/15) of the mice. However, only 25% ( 4/16) of the TM40D-Mp cells inoculated mice developed bone lesions (p<0.05) (FIG. 5B). As shown in FIG. 5C, mice that were inoculated with TM40D cells had a higher mortality rate compared to those mice that received TM40D-Mp tumor cells (P<0.05).

### EXAMPLE VIII

This example shows modulation of the pattern of bone metastatic lesions by maspin. Mice inoculated with TM40D cells or TM40D-Mp cells developed lesions with a preference in the distal femurs and proximal tibiae within 4-6 weeks (FIG. 6). The TM40D inoculated mice developed exclusively osteolytic lesions (FIG. 6B). However, the bone lesions from the TM40D-Mp inoculated mice displayed both osteosclerotic lesions and increased bone density (FIG. 6C). To evaluate the difference of bone lesions between TM40D and TM40D-Mp cells, NIH Image Software was used to measure the area decreased bone density in the right femur (inoculated tumor cells) of each mouse based on X-ray image. As shown in FIG. 6D, a significant loss of bone mass density was observed in TM40D mice as compared that with TM-Mp-inoculated mice (P=0.02).

Histological analysis of serially sectioned bone tissue by H&E staining showed that certain regions of bone marrow cavity were replaced by TM40D breast tumors (FIG. 7, C and D). This pattern of osteolytic lesion was observed in mice inoculated with TM40D tumor cells. However, new bone formation was present only in the TM40D-Mp bone tissue that had newly formed bone surrounded by the tumor cells (FIG. 7, E and F).

### EXAMPLE IX

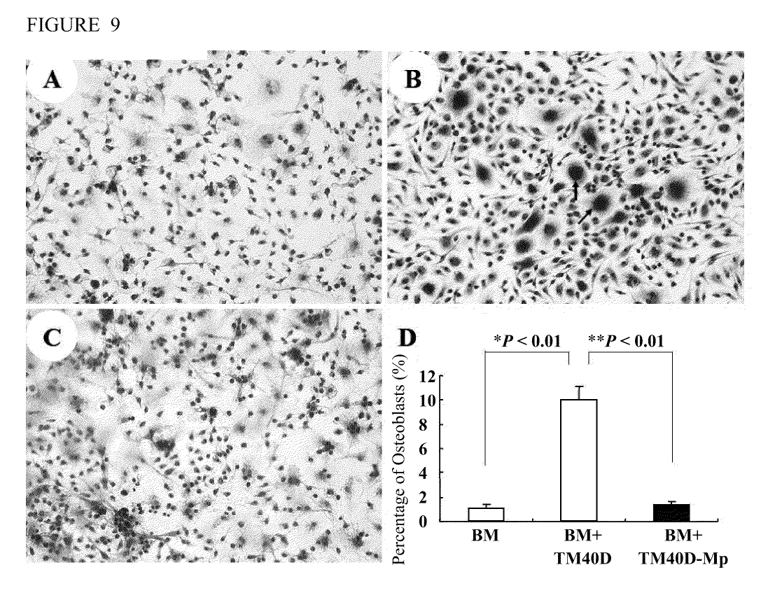

This example shows that maspin reduces osteoclast differentiation. Tumor-bone host interaction modulates bone remodeling by interfering with the differentiation of osteoblasts and osteoclasts. Osteolytic lesions are generated by the osteoclasts that are present at the boundary of bone matrix and tumor masses. An increased number of osteoclasts correlates with the increased size of osteolytic lesions. To determine whether TM40D-Mp tumors could affect osteoclast differentiation, the number of osteoclast cells in bone regions containing osteolytic lesions was compared among samples from TM40D and TM40D-Mp inoculated mice. The number of osteoclasts lining the bone surface in the TM40D group was significantly higher than that of the TM40D-Mp group (P=0.027) (FIG. 8A, B). An in vitro osteoclast cell differentiation assay was performed to further examine the effect of maspin on bone remodeling. TM40D cells or TM40D-Mp cells were co-cultured with primary mouse bone marrow cells isolated from Balb/C mice. Following 6 days in culture, cells were fixed and osteoclast-positive cells were identified by TRAP staining using a commercially available kit. TRAP positive cells with two or more nuclei were identified as mature osteoclasts (FIG. 9, A, B, and C). As shown in FIG. 9, in the absence of breast tumor cells, very few bone marrow cells differentiated into TRAP positive osteoclast cells (FIG. 9A). However, when the bone marrow cells were co-cultured with TM40D cells, osteoclast formation was strongly stimulated (FIG. 9B), whereas the number of osteoclasts in the co-culture with TM40D-Mp cells was drastically reduced (FIG. 9C). Statistical analysis confirmed that co-culture of bone marrow cells with TM40D cells significantly increased osteoclast cell differentiation (P<0.01), and the presence of maspin in TM40D-Mp cells significantly antagonized this effect (P<0.01) (FIG. 5D).

### EXAMPLE X

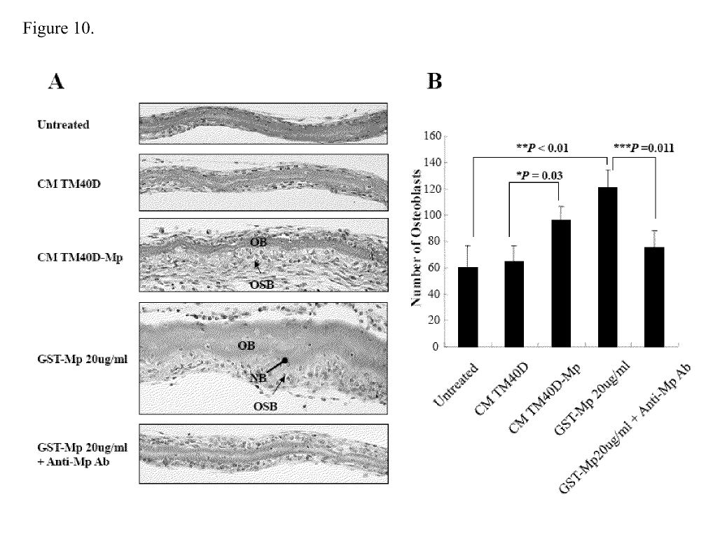

This example shows that maspin induces osteoblast cell proliferation and differentiation. To examine whether TM40D cells and TM40D-Mp cells also have different effects on osteoblasts, osteoblastic proliferation and differentiation assays were peformed by co-culture of primary mouse osteoblasts (PMOs) and TM40D cells or TM40D-Mp cells. The proliferation assay showed that TM40D cells significantly inhibited PMOs proliferation (P<0.01), while this inhibitory effect was reversed when TM40D-Mp cells were used in the assay (P<0.01) (FIG. 10 A). To confirm whether the effect is due to secreted maspin from TM40D-Mp acting on cell surface of PMOs, the cells were treated with a specific maspin antibody. Antibody treatment specifically the effect of increased proliferation by TM40D-Mp (P<0.01) (FIG. 10A). Whether maspin can affect osteoblastic cell differentiation by an assay to measure the activity of alkaline phosphatase was examined. As shown in FIG. 10B, while TM40D cells did not have an effect on the activity of alkaline phosphatase in co-cultured PMOs (P=0. 1), TM40D-Mp cells significantly induced the activity of alkaline phosphatase in PMOs (P=0.03).

### EXAMPLE XI

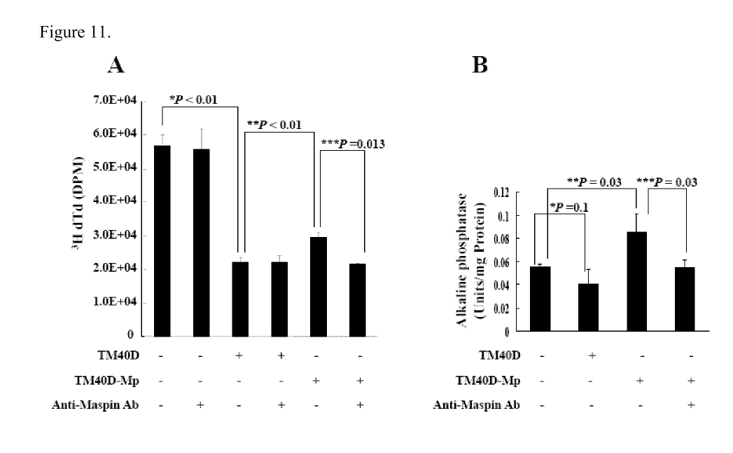

This example shows the effects of maspin on mouse calvarial organ culture. A mouse calvarial culture system was used to study the effect of maspin on new bone formation. Mouse calvariae were either treated with conditioned medium (CM) prepared from TM40D and TM40D-Mp cell culture or with recombinant GST-Mp protein for 7 days. After the treatment, calvarial samples were collected and sectioned. The sections were viewed under microscope and the images were analyzed using an osteometrics software. FIG. 11A show representative images generated from a calvaria section viewed at X 200 magnification. The number of osteoblasts in various treated calvarial samples were compared. The data showed that treatment with the CM from TM40D-Mp cell culture significantly increased the number of osteoblasts compared to the treatment with the CM from TM40D cell culture (P=0.03) (shown as FIG. 11B). To confirm that the effect observed is maspin specific, the organ culture was treated with recombinant maspin (GST-Mp). Treatment of GST-Mp at 20 μg/ml strongly increased the number of osteoblasts and new bone formation in the organ culture (P<0.01), and this effect was specifically blocked by the addition of an anti-maspin antibody (P<0.05)

### EXAMPLE XII

This example shows altered gene expression in TM40D and TM40D-Mp cells. The ability of the presence of maspin in TM40D-Mp tumor cells to alter the expression of other genes that affect the pattern of bone metastasis and the interaction between tumor cells and bone host was investigated. Several known molecules have been implicated in the modulation of the pattern of osteoclast and osteoblast lesions in bone, including OPN, PTHrP, and OPG. Semi-quantitative RT-PCR analyses were conducted to determine the levels of OPN, PTHrP, and OPG gene expression in TM40D and TM40D-Mp cells (FIG. 12A). OPN was expressed at much higher level in TM40D cells compared to that in TM40D-Mp cells (P=0.015) (FIG. 12B). There was no significant difference in the level of PTHrP and OPG expression between TM40D cells and TM40D-Mp cells (FIG. 12C and D).

### EXAMPLE XIII

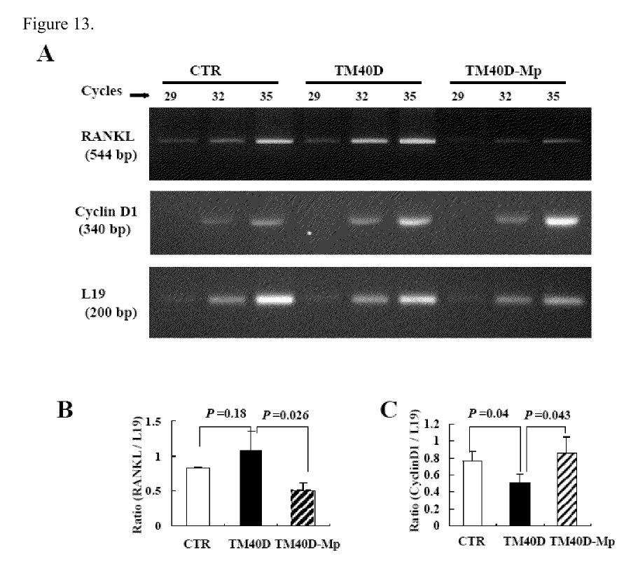

This example shows that maspin regulates RANKL and Cyclin D1 expression in osteoblastic cells. RANKL is a ligand produced by osteoblastic cells. During breast cancer metastasis, osteoblastic cells-secreted RANKL has been shown to promote osteoclast formation by mediated the interaction between osteoblastic cells and breast cancer cells (see, e.g., Traianedes K, et al., Endocrinology 1998;139(7):3178-84; herein incorporated by reference in its entirety). Per observation of a reduced osteclastic lesion in mice implanted with maspin-expressing TM40D-Mp tumor cells, experiments were conducted to determine whether maspin secreted from TM40D-Mp cells decreases the expression level of RANKL in osteoblastic cells, which causes a shift of balance from osteoclastic to osteoblastic lesion. Secondly, since maspin increases osteoblastic cell proliferation and cyclin D1 is one important factor that promotes cell cycle progression in osteoblastic cells, experiments were conducted to determine whether maspin secreted from TM40D-Mp cells regulate the expression level of cyclin D1 in osteoblastic cells. Osteblastic cells were co-cultured with TM40D-Mp and control TM40D cells and the expression level of RANKL and cyclin D1 in osteoblasts were analyzed. Osteoblast precursor MC3T3-E1 cells were treated with conditioned media (CM) collected from TM40D or TM40D-Mp cell culture. Cultured MC3T3-E1 cells were harvested for RNA extraction and subsequent RT-PCR analysis for the expression of selected genes. As shown in FIG. 13A and B, the level of RANKL mRNA from cells co-cultured with the CM of TM40D-Mp cells was significantly lower than that from cells co-cultured with the CM of TM40D cells (P<0.026). However, the level of cyclin D1 mRNA in osteoblastic cells treated with TM40D-Mp CM was significantly higher than that from the cells treated by TM40D CM (P=0.043) (FIG. 13A and C). While not limited to a particular mechanism, these results indicate that maspin regulates bone lesion pattern by decreasing RANKL expression level and by increasing cyclin D1 expression and cell proliferation rate in osteoblastic cells.

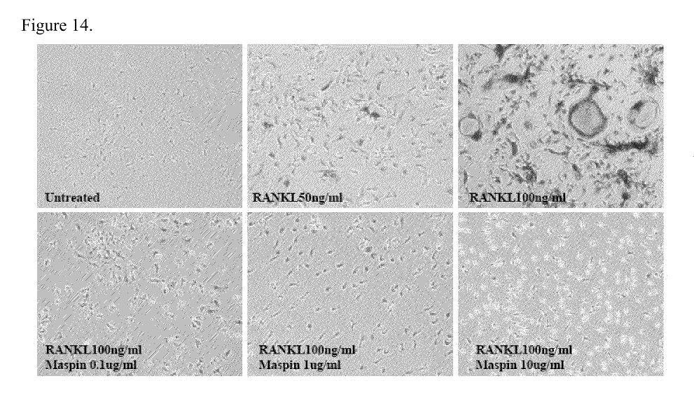

The effect of maspin on osteoclast differentiation was examined. In particular, monocytes were cultured by themselves or in the presence of RANKL ligand at 50 and 100 ng/ml for 7 days, and the differentiation of monocytes to osteclast cells measured. As shown in FIG. 14, in the presence of maspin at various dosages, the differentiation of monocytes to osteclast cells was significantly inhibited. In parituclar, at 10 μg/ml, maspin inhibited the formation of osteoclast cells. While not limited to a particular mechanism, these results indicate that maspin controls osteoclast formation through inhibition of RANKL-mediated signal transduction.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method for treating a bone formation disorder, comprising administering to a subject suffering from a bone formation disorder purified maspin.

2. The method of claim 1, wherein said bone formation disorder is osteoporosis.

3. The method of claim 1, wherein said purified maspin is provided via TM40-Mp cells.

4. The method of claim 1, wherein said purified maspin is recombinant maspin.

5. The method of claim 1, wherein said subject is a mouse.

6. The method of claim 1, wherein said subject is a human being.

7. The method of claim 1, wherein said bone formation disorder is osteoporosis, and wherein said composition is co-administered with at least one anti-osteoporosis agent.

8. A method for preventing formation of a bone formation disorder, comprising administering to a subject at risk for developing a bone formation disorder purified maspin.

9. The method of claim 8, wherein said bone formation disorder is osteoporosis.

10. The method of claim 8, wherein said purified maspin is provided via TM40-Mp cells.

11. The method of claim 8, wherein said purified maspin is recombinant maspin.

12. The method of claim 8, wherein said subject is a geriatric human being.

13. A method for promoting osteoblast cell proliferation comprising administering to a sample comprising osteoblast cells a composition comprising maspin.

14. The method of claim 13, wherein said composition comprises maspin expressing TM40-Mp cells.

15. The method of claim 13, wherein said composition comprises recombinant maspin.

16. The method of claim 13, wherein said sample is from a human being.


SEQUENCE LISTING (12 sequences)

SEQ ID NO: 1 - DNA, 28 bp, Artificial Sequence
  Synthetic
agtcgacatgagattggcagtgatttgc

SEQ ID NO: 2 - DNA, 28 bp, Artificial Sequence
  Synthetic
actcgaggcctcttctttagttgacctc

SEQ ID NO: 3 - DNA, 19 bp, Artificial Sequence
  Synthetic
gagcgcagatggatcctaa

SEQ ID NO: 4 - DNA, 21 bp, Artificial Sequence
  Synthetic
gaacttgggattttgatgctg

SEQ ID NO: 5 - DNA, 18 bp, Artificial Sequence
  Synthetic
accaaagtgaatgccgag

SEQ ID NO: 6 - DNA, 19 bp, Artificial Sequence
  Synthetic
aagaaacagcccagtgacc

SEQ ID NO: 7 - DNA, 23 bp, Artificial Sequence
  Synthetic
tcctaacagaatatcagaagaca

SEQ ID NO: 8 - DNA, 24 bp, Artificial Sequence
  Synthetic
aggcttgcctcgctgggccacatc

SEQ ID NO: 9 - DNA, 22 bp, Artificial Sequence
  Synthetic
tctacactgacaactctatccg

SEQ ID NO: 10 - DNA, 22 bp, Artificial Sequence
  Synthetic
tagcaggagaggaagttgttgg

SEQ ID NO: 11 - DNA, 21 bp, Artificial Sequence
  Synthetic
ctgaaggtcaaagggaatgtg

SEQ ID NO: 12 - DNA, 21 bp, Artificial Sequence
  Synthetic
ggacagagtcttgatgatctc